SEQUENCE LISTING (18 sequences)

SEQ ID NO: 1 - protein, 524 aa, Homo sapiens [sequence omitted]

SEQ ID NO: 2 - protein, 83 aa, Homo sapiens
SLPCDICKDVVTAAGDMLKDNATEEEILVYLEKTCDWLPKPNMSASCKEIVDSYLPVILDIIKGEMSRPGEVCSALNLCESLQ

SEQ ID NO: 3 - protein, 80 aa, Homo sapiens
GDVCQDCIQMVTDIQTAVRTNSTFVQALVEHVKEECDRLGPGMADICKNYISQYSEIAIQMMMHMQPKEICALVGFCDEV

SEQ ID NO: 4 - protein, 80 aa, Homo sapiens
SDVYCEVCEFLVKEVTKLIDNNKTEKEILDAFDKMCSKLPKSLSEECQEVVDTYGSSIPSILLEEVSPELVCSMLHLCSG

SEQ ID NO: 5 - protein, 82 aa, Homo sapiens
DGGFCEVCKKLVGYLDRNLEKNSTKQEILAALEKGCSFLPDPYQKQCDQFVAEYEPVLIEILVEVMDPSFVCLKIGACPSAH

SEQ ID NO: 6 - DNA, 1524 bp, Homo sapiens
ggcccggtccttggactgaaagaatgcaccaggggctcggcagtgtggtgccagaatgtgaagacggcgtccgactgcggggcagtgaagcactgcctgcagaccgtttggaacaagccaacagtgaaatcccttccctgcgacatatgcaaagacgttgtcaccgcagctggtgatatgctgaaggacaatgccactgaggaggagatccttgtttacttggagaagacctgtgactggcttccgaaaccgaacatgtctgcttcatgcaaggagatagtggactcctacctccctgtcatcctggacatcattaaaggagaaatgagccgtcctggggaggtgtgctctgctctcaacctctgcgagtctctccagaagcacctagcagagctgaatcaccagaagcagctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcggctgcctgcactgaccgttcacgtgactcagccaaaggacggtggcttctgcgaagtgtgcaagaagctggtgggttatttggatcgcaacctggagaaaaacagcaccaagcaggagatcctggctgctcttgagaaaggctgcagcttcctgccagacccttaccagaagcagtgtgatcagtttgtggcagagtacgagcccgtgctgatcgagatcctggtggaggtgatggatccttccttcgtgtgcttgaaaattggagcctgcccctcggcccataagcccttgttgggaactgagaagtgtatatggggcccaagctactggtgccagaacacagagacagcagcccagtgcaatgctgtcgagcattgcaaacgccatgtgtggaac

SEQ ID NO: 7 - DNA, 243 bp, Homo sapiens
tctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacg

SEQ ID NO: 8 - DNA, 6619 bp, Artificial
  Vector sequence with PSAP insert
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtgacgcggcccagccggccggcccggtccttggactgaaagaatgcaccaggggctcggcagtgtggtgccagaatgtgaagacggcgtccgactgcggggcagtgaagcactgcctgcagaccgtttggaacaagccaacagtgaaatcccttccctgcgacatatgcaaagacgttgtcaccgcagctggtgatatgctgaaggacaatgccactgaggaggagatccttgtttacttggagaagacctgtgactggcttccgaaaccgaacatgtctgcttcatgcaaggagatagtggactcctacctccctgtcatcctggacatcattaaaggagaaatgagccgtcctggggaggtgtgctctgctctcaacctctgcgagtctctccagaagcacctagcagagctgaatcaccagaagcagctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcggctgcctgcactgaccgttcacgtgactcagccaaaggacggtggcttctgcgaagtgtgcaagaagctggtgggttatttggatcgcaacctggagaaaaacagcaccaagcaggagatcctggctgctcttgagaaaggctgcagcttcctgccagacccttaccagaagcagtgtgatcagtttgtggcagagtacgagcccgtgctgatcgagatcctggtggaggtgatggatccttccttcgtgtgcttgaaaattggagcctgcccctcggcccataagcccttgttgggaactgagaagtgtatatggggcccaagctactggtgccagaacacagagacagcagcccagtgcaatgctgtcgagcattgcaaacgccatgtgtggaaccatcaccatcaccatcactagctcgaggagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 5338 bp, Artificial
  Vector sequence with Saposin C insert
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtgacgcggcccagccggcctctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcatcaccatcaccatcactagctcgaggagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - DNA, 32 bp, Artificial
  oligonulceotide primer
cggggtaccaccatgtacgccctcttcctcct

SEQ ID NO: 11 - DNA, 63 bp, Artificial
  oligonucleotide primer
tatgatatcctaatggtgatggtgatggtggttccacacatggcgtttgcaatgctcgacagc

SEQ ID NO: 12 - DNA, 36 bp, Artificial
  oligonucleotide primer
aaagcggcccagccggccggcccggtccttggactg

SEQ ID NO: 13 - DNA, 37 bp, Artificial
  oligonucleotide primer
aaagcggcccagccggccatgtacgccctcttcctcc

SEQ ID NO: 14 - DNA, 50 bp, Artificial
  oligonucleotide primer
ccgctcgagctagtgatggtgatggtgatggttccacacatggcgtttgc

SEQ ID NO: 15 - DNA, 39 bp, Artificial
  oligonucleotide primer
aaagcggcccagccggcctctgatgtttactgtgaggtg

SEQ ID NO: 16 - DNA, 49 bp, Artificial
  oligonucleotide primer
ccgctcgagctagtgatggtgatggtgatgcgtgccagagcagaggtgc

SEQ ID NO: 17 - protein, 520 aa, Artificial
  Recombinant human PSAP
DAAQPAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSIPSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWNHHHHHH

SEQ ID NO: 18 - protein, 92 aa, Artificial
  Recombinant human Saposin C
DAAQPASDVYCEVCEFLVKEVTKLIDNNKTEKEILDAFDKMCSKLPKSLSEECQEVVDTYGSSIPSILLEEVSPELVCSMLHLCSGHHHHHH

(19) United States
(12) Patent Application Publication
(10) Pub. No.: US 2010/0021997 A1
(43) Pub. Date: Jan. 28, 2010

(54) Biologically active recombinant human saposin C and PSAP

(76) Inventors: Shahriar Koochekpour, Metairie, LA (US); Siyi Hu, Metairie, LA (US)

Correspondence Address: Adams and Reese LLP, Suite 4400, 1221 McKinney Street, Houston, TX, 77010, US

(21) Appl. No.: 12/378,910

(22) Filed: Feb. 20, 2009

Related U.S. Application Data
(60) Provisional application No. 61/030,307, filed on Feb. 21, 2008.

(51) Int. Cl. C12N 5/10; C12N 5/07; C07K 14/00

(52) U.S. Cl. 435/358; 530/395

(57) ABSTRACT

The invention relates to the establishment of expression vectors replicable in a mammalian cell, including a DNA sequence encoding human prosaposin (PSAP) or Saposin C, operably linked with regulatory DNA capable of effecting the expression of the PSAP and Saposin C DNA coding sequence in the cells. In addition, the invention relates to mammalian cells (CHO-K1) stably transfected with such expression vectors and methods of producing human Saposin C or PSAP, including transfecting a mammalian cell with expression vectors, maintaining and propagating the cell in culture medium, and isolating soluble rh-Saposin C or rh-PSAP from the medium. Purified rh-Saposin C and rh-PSAP are useful as reagents for studying biological responses and signal transduction mechanism in benign or malignant cells, as standard proteins in diagnosis or screening of benign or malignant diseases, or as medicine for the treatment of diseases or disorders affecting musculoskeletal system or peripheral and central nervous system.

### CROSS-REFERENCE TO RELATED APPLICATIONS

The present application claims the benefit of prior U.S. Provisional Application No. 61/030,307, filed Feb. 21, 2008, which is hereby incorporated by reference.

### STATEMENT REGARDING FEDERALLY SPONSORED RESEARCH OR DEVELOPMENT

The invention was made with U.S. Government support under contract no. NIH/NCRR 1 P20 RR021970 (01) awarded by the National Institutes of Health. The U.S. Government has certain rights in the invention.

### THE NAMES OF THE PARTIES TO A JOINT RESEARCH AGREEMENT

Not applicable.

### INCORPORATION-BY-REFERENCE OF MATERIAL SUBMITTED ON COMPACT DISC

The Sequence Listing, which is a part of the present disclosure and is submitted in conformity with 37 CFR [symbol] 1.821-1.825, includes a computer readable form and a written sequence listing comprising nucleotide and/or amino acid sequences of the present invention. The sequence listing information recorded in computer readable form (created 19 Feb. 2009; filename: SEQUENCE LISTING.txt; size: 32.2 KB; size on disk: 33.0 KB) is identical to the written sequence listing below. The subject matter of the Sequence Listing is incorporated herein by reference in its entirety.

### BACKGROUND OF THE INVENTION

1. Field of the Invention

The present invention is related to [subj matter under 424 definition] which subject matter contains a protein or its reaction product (e.g., peptides, etc.) wherein the protein molecule is not degrated to the constitutent amino acids. More specifically, the invention is related to subject matter wherein a peptide chain has 25 or more peptide units in an uninterrupted chain. In particular, the present invention is related to recombinant human (“rh”) prosaposin (PSAP) and rh-Saposin C, and to cells useful for expressing rh-PSAP and rh-Saposin C.

2. Description of Related Art

Prosaposin (PSAP; SEQ ID NO:1) is a highly conserved glycoprotein with 524 amino acids and an approximate molecular weight of 65 to 72 kilodaltons (kDa). See, e.g., Kishimoto Y, Hiraiwa M, O'Brien J S. Saposins: structure, function, distribution, and molecular genetics. J Lipid Res. 33, 1255-1267, 1999; PubMed ID: 1402395. PSAP is the precursor of four small lysosomal proteins (Saposin A, SEQ ID NO:2; Saposin B, SEQ ID NO:3; Saposin C, SEQ ID NO:4; and Saposin D, SEQ ID NO:5, each between 8 and 13 kDa) that are required for intracellular degradation of certain sphingolipids. See Kishimoto Y, 1999. Proteolytic cleavage of PSAP precursor, mediated by lysosomal cysteine protease-cathepsin D, leads to individual mature saposin proteins (acidic glycoproteins). See Kishimoto Y, 1999. PSAP and saposin proteins also exist as soluble extracellular mature proteins in tissue culture supernatant, serum, prostatic secretions, cerebrospinal fluid, seminal fluid, milk and serum. See, e.g., Morimoto S, Yamamoto Y, O'Brien J S, Kishimoto Y. Determination of saposin proteins (sphingolipid activator proteins) in human tissues. Anal Biochem. 190, 154-157, 1990; PubMed ID: 2127157. Morales C R, El-Alfy M, Zhao Q, Igdoura S A. Expression and tissue distribution of rat sulfated glycoprotein-1 (prosaposin). J Histochem Cytochem. 44, 327-337, 1996; PubMed ID: 8601692.

PSAP and individual saposin proteins are expressed by a wide variety of cell types originating from ectodermal, mesodermal, and endodermal germ layers including but not limited to lung, skin, fibroblast, stromal cells, bone, smooth muscle, skeletal muscle, cardiac muscle, placenta, red and white blood cells, pancreas, placenta, lymphoreticular system (spleen, thymus, liver), micro and macrovascular system, genitourinary system (e.g., prostate, testes, seminal vesicles), central and peripheral nervous system, etc. See, e.g., Kishimoto Y, 1999. Morimoto S, 1990. Koochekpour S. PSAP (Prosaposin (variant Gaucher disease and variant metachromatic leukodystrophy). Atlas Genet Cytogenet Oncol Haematol. 10, 370-384, 2006; available at: http://AtlasGeneticsOncology.org/Genes/PSAPID42980ch10q22.html. PSAP and saposins are predominantly expressed in neuroglial-derived tissues as compared to all other normal cell types in the mammalian system. PSAP is overexpressed in a number of malignant or metastatic cell types derived from prostate, breast, lung, brain, and lymphoid tissues. See, e.g., Campana W M, O'Brien J S, Hiraiwa M, Patton S. Secretion of prosaposin, a multifunctional protein, by breast cancer cells. Biochim Biophys Acta. 1427, 392-400, 1999; PubMed ID: 10350655. Koochekpour S, Zhuang, Yu-Jun, Beroukhim R, Hssieh G L, Hofer M D, Zhau H E, Hiraiwa M, Pattan D, Ware J L, Luftig R, Sandhoff K, Sawyers C L, Pienta K J, Rubin M A, Vessella R L, Sellers W R, and Sartor O. Amplification and Overexpression of Prosaposin in Prostate Cancer and Other Malignant Cells. Genes, Chromosomes & Cancer. 44, 351-364, 2005; PubMed ID: 16080200.

Prosaposin is a bi-functional molecule. As the precursor of intracellular lysosomal saposin proteins, it is involved in sphingolipid hydrolysis activity, as a secreted soluble protein, it has neurotrophic activities. See, e.g., O'Brien J S, Carson G S, Seo H C, Hiraiwa M, Kishimoto Y. Identification of prosaposin as a neurotrophic factor. Proc Natl Acad Sci USA. 91, 95963-95966, 1994; PubMed ID: 7937812. O'Brien J S, Carson G S, Seo H C, Hiraiwa M, Weiler S, Tomich J M, Barranger J A, Kahn M, Azuma N, Kishimoto Y. Identification of the neurotrophic factor sequence of prosaposin. FASEB J. 9, 681-685, 1995; PubMed ID: 7768361. Campana W M Hiraiwa M, Addison K C, O'Brien J S. Induction of MAPK phosphorylation by prosaposin and prosaptide in PC12 cells. Biochem Biophys Res Commun. 229, 706-712, 1996; PubMed ID: 8954961. Hiraiwa M, Taylor E M, Campana W M, Darin S J, O'Brien J S. Cell death prevention, mitogen-activated protein kinase stimulation, and increased sulfatide concentrations in Schwann cells and oligodendrocytes by prosaposin and prosaptides. Proc Natl Acad Sci USA. 94, 4778-4781, 1997; PubMed ID: 9114068. Campana W M, Hiraiwa M, O'Brien J S. Prosaptide activates the MAPK pathway by a G-protein-dependent mechanism essential for enhanced sulfatide synthesis by Schwann cells. FASEB J. 12, 307-314, 1998; PubMed ID: 9506474. Campana W M, Darin S J, O'Brien J S. Phosphatidylinositol 3-kinase and Akt protein kinase mediate IGF-I- and prosaptide-induced survival in Schwann cells. J Neurosci Res. 57, 332-341, 1999; PubMed ID: 10412024. Misasi R, Sorice M, Di Marzio L, Campana N M, Molinari S, Cifone M G, Pavan A, Pontieri G M, O'Brien J S. Prosaposin treatment induces PC12 entry in the S phase of the cell cycle and prevents apoptosis: activation of ERKs and sphingosine kinase. FASEB J. 15, 467-474, 2001; PubMed ID: 11156962. Morales C R, Badran H. Prosaposin ablation inactivates the MAPK and Akt signaling pathways and interferes with the development of the prostate gland. Asian J. Androl. 5, 57-63, 2003; PubMed ID: 12647005. Koochekpour S, Sartor O, Lee T J, Zieske A, Patten D Y, Hiraiwa M, Sandhoff K, Remmel N, Minokadeh A. Prosaptide TX14A stimulates growth, migration, and invasion and activates the Raf-MEK-ERK-RSK-Elk-1 signaling pathway in prostate cancer cells. Prostate. 61, 114-123, 2004; PubMed ID: 15305334. Lee T J, Sartor O, Luftig R B, Koochekpour S. Saposin C promotes survival and prevents apoptosis via PI3K/Akt-dependent pathway in prostate cancer cells. Mol. Cancer. 3, 31-44, 2004; PubMed ID: 15548330. Koochekpour S, Sartor O, Hiraiwa M, Lee T J, Rayford W, Remmel N, Sandhoff K, Minokadeh A, Patten D Y. Saposin C stimulates growth and invasion, activates p42/44 and SAPK/JNK signaling pathways of MAPK and upregulates uPA/uPAR expression in prostate cancer and stromal cells. Asian J. Androl. 7, 147-158, 2005; PubMed ID: 15897971. Huwiler A, Kolter T, Pfeilschifter J, Sandhoff K. Physiology and pathophysiology of sphingolipid metabolism and signaling. Biochim Biophys Acta. 1485, 63-99, 2000; PubMed ID: 10832090. Column chromatography data indicated the formation of stable complexes between PSAP/saposins and several gangliosides. It has been suggested that PSAP functions as a sphingolipid binding protein and on the cell surface, complex formation between PSAP and gangliosides may suggests a role for this molecule in ganglioside function. Saposins A-D function as co-activator proteins for intracellular lysosomal degradation of sphingolipids. See, e.g., Qi X, Grabowski G A. Molecular and cell biology of acid beta-glucosidase and prosaposin. Prog Nucleic Acid Res Mol. Biol. 66, 203-239, 2001; PubMed ID: 11051765. Sandhoff K, Kolter T. Biosynthesis, and degradation of mammalian glycosphingolipids. Philos Trans R Soc Lond B Biol Sci. 358, 847-861, 2003; PubMed ID: 12803917. Saposin A and C are involved in hydrolysis of glucosylceramide and galactosylceramide. Saposin B stimulates galacto-cerebroside sulfate hydrolysis, GM1 ganglioside, and globotriaosylceramide. Saposin C is the activator of sphingomyelin phosphodiesterase. While several members of CD1 proteins are involved in lipid presentation to T cells, prosaposin-deficient mice exhibit certain defects in CD1d-mediated antigenic presentation, suggesting that saposins are involved in mobilization of lipid monomers from the lysosomal membrane and their association with CD1d. In addition, prosaposin-deficient fibroblasts transfected with another member of CD1 family (CD1b) also failed to activate lipid-specific T lymphocytes. See, e.g., Kang S J, Cresswell P. Saposins facilitate CD1d-restricted presentation of an exogenous lipid antigen to T cells. Nat. Immunol. 5, 175-181, 2004; PubMed ID: 14716312. Winau F, Schwierzeck V, Hurwitz R, Remmel N, Sieling P A, Modlin R L, Porcelli S A, Brinkmann V, Sugita M, Sandhoff K, Kaufmann S, Schaible U E. Saposin C is required for lipid presentation by human CD1b. Nat. Immunol. 5, 169-174, 2004. Erratum in: Nat. Immunol. 5, 344, 2004; PubMed ID: 14716313. Zhou D, Cantu C 3rd, Sagiv Y, Schrantz N, Kulkarni A B, Qi X, Mahuran D J, Morales C R, Grabowski G A, Benlagha K, Savage P, Bendelac A, Teyton L. Editing of CD1d-bound lipid antigens by endosomal lipid transfer proteins. Science. 303, 523-527, 2004; PubMed ID: 14684827. Prosaposin's bi-functional nature may be suggestive of potential implications for Saposin C or perhaps PSAP in the recognition of tumor antigens.

Several reports have identified in vitro or in vivo neurotrophic/neuroprotective activities for PSAP or Saposin C. In addition, a number of linear 5 to 22 amino acid sequences called prosaptides (e.g., D5, TX14A) located at the amino terminal region of Saposin C domain of PSAP demonstrate in vitro and/or in vivo neurotrophic activities. See, e.g., Henseler M, Klein A, Glombitza G J, Suziki K, Sandhoff K. Expression of the three alternative forms of the sphingolipid activator protein precursor in baby hamster kidney cells and functional assays in a cell culture system. J Biol. Chem. 271, 8416-8423, 1996; PubMed ID: 8626540. Kotani Y, Matsuda S, Wen T C, Sakanaka M, Tanaka J, Maeda N, Kondoh K, Ueno S, Sano A. A hydrophilic peptide comprising 18 amino acid residues of the prosaposin sequence has neurotrophic activity in vitro and in vivo. J. Neurochem. 66, 2197-2200, 1996; PubMed ID: 8780053. The neurotrophic activities of PSAP or its biologically active molecular derivatives (i.e., Saposin C, prosaptides) include growth, development, and maintenance of the peripheral and central nervous systems, neuronal regeneration and plasticity, stimulation of neurite outgrowth, attenuation of loss of muscle mass following nerve injury, stimulation of neuroblastoma cell proliferation, protection of neurons from programmed cell-death (apoptosis), and activation of cell survival signaling pathways such (but not limited to) as MAPK and PI3K/Akt pathways.

By using immunohistochemical staining, intense staining of PSAP was observed in adult rat skeletal, smooth, and cardiac muscle cells with normal innervation. See e.g., Rende M, Brizi E, Donato R, Provenzano C, Bruno R, Misisin A P, Garrett R S, Calcutt N A, Campana W M, O'Brien J S. Prosaposin is immunolocalized to muscle and prosaptides promote myoblast fusion and attenuate loss of muscle mass after nerve injury. Muscle Nerve. 24, 799-808, 2001; PubMed ID: 11360264. Following nerve injury and muscle denervation, PSAP immunostaining was decreased. These data suggest that, in addition to neurotrophic activities, PSAP or its active molecular derivatives has myotrophic activity.

Overall, PSAP, Saposin C, and PSAP-originated oligopeptides demonstrate a number of therapeutic applications, including promoting biofunctional recovery following toxic or physical injury to nervous system, myocardial hypoxic injury, and degenerative or inherited diseases of central or peripheral nervous system. See, e.g., Jolivalt C G, Ramos K M, Herbetsson K, Esch F S, Calcutt N A. Therapeutic efficacy of prosaposin-derived peptide on different models of allodynia. Pain 121, 14-21, 2006; PubMed ID: 16480831. Wagner R, Myers R R, O'Brien, J S. Prosaptide prevents hyperalgesia and reduces peripheral TNFR1 expression following TNF nerve injection. Neuroreport. 9, 2827-2831, 1998; PubMed ID: 9760128. Campana W M, Mohiuddin L, Misasi R, O'Brien J S, Calcutt N A. Prosaposin-derived peptides enhanced sprouting of sensory neurons in vitro and induced sprouting at motor endplates in vivo. J Peripher Nerv Syst. 5, 126-130, 2000; PubMed ID: 11442168. See also: U.S. Pat. No. 5,571,787, issued 5 Nov. 1996. In addition, by inducing neurite outgrowth and stimulating myelination, PSAP, Saposin C, and PSAP-derived peptides are able to inhibit and reduce the consequences of demyelinating diseases. See, e.g., Hiraiwa M, Campana W M, Mizisin A P, Mohiuddin L, O'Brien J S. Prosaposin: a myelinotrophic protein that promotes expression of myelin constituents and is secreted after nerve injury. Glia. 26, 353-360, 1999; PubMed ID: 10383054.

In malignant cells and tissues, several classic reports have indicated a pluripotent regulatory role for Saposin C and PSAP in prostate cancer, with potential involvement in prostate carcinogenesis or progression toward metastatic or androgen-independent state. In addition, under serum-starvation stress, PSAP and/or its active molecular derivatives (Saposin C or TX14A): a) stimulate prostate cancer cell growth, motility, and invasion; b) upregulate proteolytic enzyme uPA/uPAR expression; c) activate the p42/44 MAPK (Raf-MEK-ERK-RSK-Elk-1 signaling cascade), p38 MAPK, and SAPK/JNK family members of the MAPK superfamily and PI3K/Akt signaling pathways; and d) protect cells from apopototic cell death induction by etoposide via modulation of caspase-3, -7, and -9 expression/activity and/or the PI3K/Akt signaling pathway activation. Although several reports have indicated a potential role for PSAP in a variety of cancers (e.g., breast, pancreas, brain, lung, lymphoma) in general, the expression and biological significance of PSAP or Saposin C in cancer biology requires further investigation.

Deficiencies of PSAP and/or saposins B, C, or D are reported to be responsible for a number of clinical diseases. The clinical features in patients with total PSAP deficiency (combined SAP deficiency) are reported to be similar to those in Gaucher disease type 2, which present with acute infantile neuronopathic symptoms, abnormally large visceral organs, deteriorating general physical condition, and death in the first two years of life. Saposin A deficiency as a disease entity has not yet been reported. However, mice with mutated saposin A demonstrate a phenotype similar to late-onset Krabbe disease. Patients with saposin B deficiency show clinical symptoms similar to those associated with Metachromatic leukodystrophy (MLD). Saposin C deficient patients present with clinical findings similar to Gaucher disease type 3. Saposin D mutation in a mouse model has shown progressive polyuria and ataxia and accumulation of ceramide in brain and kidney. Accumulation of saposins (up to 80-fold over normal levels) are detected in spleen, liver, and brain of individuals affected with lysosomal storage diseases (LSD) such as Gaucher disease, Niemann-Pick disease (type 1), fucosidosis, Tay-Sachs disease, and Sandhoff disease. Analysis of plasma levels of saposins in patients with LSD disorders often reveals an increase in saposin A-D levels.

Total prosaposin deficiency leads to a lethal phenotype in both humans and mice. Mice with homozygous inactivation of prosaposin gene (PSAP−/−) demonstrated clinicopathologic signs similar those of human patients with total PSAP deficiency. Among these features was intrauterine or early neonatal death in PSAP−/− mice. In other mice, severe developmental abnormalities in the nervous system and male reproductive system was detected. Neuroembryological developmental abnormalities presented as muscular weakness, trembling or shakiness of head, and ataxia of the limbs and progressed to severe weakness and shaking of head and trunk. After 4 weeks they developed seizures and persistent tonic epilepsy, and finally died at the age of 35 days. Evidence of lysosomal storage disease was also detected by abnormal accumulation of ceramide in brain, liver, and kidney, and storage of gangliosides and ceramide and hypomyelination of the brain.

### BRIEF SUMMARY OF THE INVENTION

The present invention features, in one aspect, a cell transfected with a mammalian expression vector, wherein said vector provides for expression of recombinant human (“rh”) PSAP, and wherein said rh-PSAP is extracellularly secreted. In a preferred embodiment of this aspect, the cell is a CHO-K1 cell, and the CHO-K1 cell is stably transfected. Also preferably, in this embodiment, the vector bears, in 5′ to 3′ direction, an Ig kappa-chain V-J2-C signal peptide sequence, SEQ ID NO:6, and a hexa-histidine epitope. Preferably, the vector is a pSecTag2 vector into which SEQ ID NO:6 has been cloned. More preferably, the vector is SEQ ID NO:8.

The present invention features, in another aspect, a cell transfected with a mammalian expression vector, wherein said vector provides for expression of recombinant human (“rh”) Saposin C, and wherein said rh-Saposin C is extracellularly secreted. In a preferred embodiment of this aspect, the cell is a CHO-K1 cell, and the CHO-K1 cell is stably transfected. Also preferably, in this embodiment, the vector bears, in 5′ to 3′ direction, an Ig kappa-chain V-J2-C signal peptide sequence, SEQ ID NO:7, and a hexa-histidine epitope. Preferably, the vector is a pSecTag2 vector into which SEQ ID NO:7 has been cloned. More preferably, the vector is SEQ ID NO:9.

In another aspect, the present invention features an isolated recombinant human PSAP comprising the amino acid sequence of SEQ ID NO:17. Preferably, in this aspect, the isolated rh-PSAP is an N-linked glycoprotein and bears a C-terminal 6×His epitope. Even more preferably, in this aspect, the isolated rh-PSAP is a sialylated N-linked glycoprotein and bears a C-terminal 6×His epitope.

In another aspect, the present invention features an isolated recombinant human Saposin C comprising the amino acid sequence of SEQ ID NO:18. Preferably, in this aspect, the isolated rh-Saposin C is an N-linked glycoprotein and bears a C-terminal 6×His epitope. Even more preferably, in this aspect, the isolated rh-Saposin C is a sialylated N-linked glycoprotein and bears a C-terminal 6×His epitope.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements.

FIG. 1 is a schematic representation of rh-PSAP and rh-Saposin C expression vectors. The rh-PSAP (SEQ ID NO:6) or rh-Saposin C (SEQ ID NO:7) cDNA sequence was cloned into the SfiI and XhoI sites of pSecTag2A vector in frame with the N-terminal Ig-kappa leader sequence under the control of human cytomegalovirus (CMV) immediate-early promoter.

FIG. 2 is a Western blot analysis of rh-PSAP expression in transiently-transfected human embryonic kidney (HEK) 293T/17 cells. 293T/17 cells were transiently transfected with different rh-PSAP expression vectors. For each construct, 10 μg of supernatant protein was separated in a 4-20% gradient polyacrylamide gel and transferred to PVDF membrane. The membrane was blotted with mouse anti-PSAP antibody. As a positive control, we used 2 μl of purified human milk PSAP. 293T/17 transfected clones with empty vectors were used for comparison. Solid arrow shows PSAP protein.

FIG. 3 is a Western blot analysis of rh-PSAP and rh-Saposin C expression in stably-transfected CHO-K1 clones. For each stable clone, 10 μg of supernatant protein was separated in a 4-20% gradient polyacrylamide gel and transferred to PVDF membrane. The membrane was blotted with goat anti-Saposin C polyclonal antibody, which can also detect PSAP. PC3 supernatant was used as control for native human PSAP. Solid arrows show rh-PSAP or rh-Saposin C. Dashed arrows around 30 and 55 kDa show cleaved PSAP protein products.

FIG. 4 shows SDS-PAGE and Coomassie blue staining analysis for rh-PSAP and rh-Saposin C expression and purification. 40 μl of culture supernatant or 20 μl imidazole washing or elution fraction of rh-PSAP and rh-Saposin C was separated in 4-20% tris-glycine gel. Solid arrow indicate rh-PSAP (˜70 kDa) or rh-Saposin C (˜10 kDa).

FIG. 5 shows SDS-PAGE and silver staining of final purified rh-PSAP and rh-Saposin C. 2 μg rh-PSAP and 0.1 μg bovine serum albumin (BSA) was separated in 4-20% gel (A). Various amount of baculovirus-infected insect cells expressed rh-Saposin C was separated in 10-20% gel. Solid arrows indicate rh-Saposin C (˜10 kDa) or rh-PSAP (˜70 kDa). Dashed arrows indicate extracellularly cleaved products of rh-PSAP protein.

FIG. 6 shows a Western blot analysis of final purified rh-PSAP and rh-Saposin C Various amounts of purified rh-PSAP were separated in 4-20% gel and blotted with mouse anti-PSAP antibody. For Saposin C, various amounts of rh-Saposin C (200-400 ng) were separated in a 10-20% gradient gel and blotted with goat anti-Saposin C antibody. Solid arrows indicate rh-Saposin C (˜10 kDa) or rh-PSAP (˜70 kDa). Dashed arrows indicate the presence of extracellularly cleaved products of rh-PSAP protein.

FIG. 7 illustrates the stimulation of BrdU incorporation as an indicator of cell proliferation by various concentrations of rh-PSAP, or rh-Saposin C, indicating that both pSecTag2A-recombinant proteins are biologically active in mammalian cell types (i.e., PC-3—ATCC No. CRL-1435—and PrSt—prostate stromal—cells). Baculovirus-expressed rh-Saposin C was used as a positive control for cell growth.

FIG. 8 illustrates the stimulation of cell migration by various concentrations of rh-Saposin C and rh-PSAP on prostate stromal and cancer cell line, indicating that these purified proteins are biologically active. Baculovirus-expressed recombinant Saposin C was used a positive (active) control.

### DETAILED DESCRIPTION OF THE INVENTION

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

In the following description, terms relating to recombinant DNA technology are used. The following definitions are provided to give a clear understanding of the specification and appended claims.

As used herein, the term “PSAP” means prosaposin, and the term “rh-PSAP” means recombinant human prosaposin.

By “gene” is meant a nucleic acid (e.g., deoxyribonucleic acid, or “DNA”) sequence that comprises coding sequences necessary for the production of a polypeptide or precursor (e.g., messenger RNA, or “mRNA”). The polypeptide may be encoded by a full length coding sequence or by any portion of the coding sequence, so long as the desired activity or functional properties (e.g., enzymatic activity, ligand binding, signal transduction, etc.) are retained. The term also encompasses the coding region of a structural gene and the sequences located adjacent to the coding region on both the 5′ and 3′ ends, for a distance of about 1 kb on either end, such that the gene is capable of being transcribed into a full-length mRNA. The sequences located 5′ of the coding region and which are present on the mRNA are referred to as 5′ untranslated sequences, and form the 5′ untranslated region (5′ UTR). The sequences located 3′ or downstream of the coding region and which are present on the mRNA are referred to as 3′ non-translated sequences, and form the 3′ untranslated region (3′ UTR). The term “gene” encompasses both cDNA and genomic forms of a gene. The genomic form or clone of a gene usually contains the coding region interrupted with non-coding sequences termed “introns” (also called “intervening regions” or “intervening sequences”). Introns are segments of a gene which are transcribed into nuclear RNA (hnRNA); introns may contain regulatory elements such as enhancers. Introns are removed or “spliced out” from the nuclear or primary transcript, and therefore are absent from the mRNA transcript. mRNA functions during translation to specify the sequence or order of amino acids in a nascent polypeptide.

By “nucleotide” is meant a monomeric structural unit of nucleic acid (e.g., DNA or RNA) consisting of a sugar moiety (a pentose: ribose, or deoxyribose), a phosphate group, and a nitrogenous heterocyclic base. The base is linked to the sugar moiety via a glycosidic bond (at the 1′ carbon of the pentose ring) and the combination of base and sugar is called a nucleoside. When the nucleoside contains a phosphate group bonded to the 3′ or 5′ position of the pentose, it is referred to as a nucleotide. When the nucleotide contains one such phosphate group, it is referred to as a nucleotide monophosphate; with the addition of two or three such phosphate groups, it is called a nucleotide diphosphate or triphosphate, respectively. The most common, nucleotide bases are derivatives of purine or pyrimidine, with the most common purines being adenine and guanine, and the most common pyrimidines being thymidine, uracil, and cytosine. A sequence of operatively linked nucleotides is typically referred to herein as a “base sequence” or “nucleotide sequence” or “nucleic acid sequence,” and is represented herein by a formula whose left-to-right orientation is in the conventional direction of 5′-terminus to 3′-terminus. A “test nucleic acid sequence” is a nucleic acid sequence used according to the methods of the present invention to measure or test interaction between said nucleic acid sequence and a protein. The test nucleic acid sequence may be a genomic DNA fragment.

DNA molecules are said to have “5′ ends” and “3′ ends” because mononucleotides are joined to make oligonucleotides in a manner such that the 5′ phosphate of one mononucleotide pentose ring is attached to the 3′ oxygen of its neighbor in one direction, via a phosphodiester linkage. Therefore, an end of an oligonucleotide is referred to as the “5′ end” if its 5′-phosphate is not linked to the 3′ oxygen of a mononucleotide pentose ring. Alternatively, it is the “3′ end” if its 3′ oxygen is not linked to a 5′ phosphate of a subsequent mononucleotide pentose ring. These ends are also referred to as “free” ends because they are not linked to upstream or downstream mononucleotides, respectively. A double stranded nucleic acid molecule may also be said to have 5′- and 3′ ends, wherein the “5′” refers to the end containing the accepted beginning of the particular region, gene, or structure, and the “3′” refers to the end downstream of the 5′ end. A nucleic acid sequence, even if internal to a larger oligonucleotide, may also be said to have 5′ and 3′ ends, although these ends are not free ends. In such a case, the 5′ and 3′ ends of the internal nucleic acid sequence refer to the 5′ and 3′ ends that said fragment would have were it isolated from the larger oligonucleotide. In either a linear or circular DNA molecule, discrete elements may be referred to as being “upstream” or 5′ of the “downstream” or 3′ elements. Ends are said to “compatible” if: a) they are both blunt or contain complementary single strand extensions (such as that created after digestion with a restriction endonuclease); and b) at least one of the ends contains a 5′ phosphate group. Compatible ends are therefore capable of being ligated by a double stranded DNA ligase (e.g., T4 DNA ligase) under standard conditions. Nevertheless, blunt ends may also be ligated.

By “promoter” is meant a DNA sequence usually found at the 5′ region of a gene, proximal to the start codon. Transcription of an adjacent gene is initiated at the promoter region. If the promoter is an inducible promoter, the rate of transcription increases in response to an inducing agent.

By “operably linked” is meant that nucleic acid sequences or proteins are operably linked when placed into a functional relationship with another nucleic acid sequence or protein. For example, a promoter sequence is operably linked to a coding sequence if the promoter promotes transcription of the coding sequence. As a further example, a repressor protein and a nucleic acid sequence are operably linked if the repressor protein binds to the nucleic acid sequence. Additionally, a protein may be operably linked to a first and a second nucleic acid sequence if the protein binds to the first nucleic acid sequence and so influences transcription of the second, separate nucleic acid sequence. Generally, “operably linked” means that the DNA sequences being linked are contiguous, although they need not be, and that a gene and a regulatory sequence or sequences (e.g., a promoter) are connected in such a way as to permit gene expression when the appropriate molecules (e.g., transcriptional activator proteins—transcription factors—or proteins which include transcriptional activator domains) are bound to the regulatory sequence or sequences.

By “protein” is meant a sequence of amino acids of any length, constituting all or a part of a naturally-occurring polypeptide or peptide, or constituting a non-naturally occurring polypeptide or peptide (e.g., a randomly generated peptide sequence or one of an intentionally designed collection of peptide sequences).

By “expression” or “gene expression” is meant transcription (e.g., from a gene) and, in some cases, translation of a gene into a protein, or “gene product.” In the process of expression, a DNA chain coding for the sequence of gene product is first transcribed to a complementary RNA, which is often a messenger RNA, and, in some cases, the transcribed messenger RNA is then translated into the gene product—a protein. The terms are also used to mean the degree to which a gene is active in a cell or tissue, measured by the amount of mRNA in the tissue and/or the amount of protein expressed.

As used herein, the terms “vector” or “plasmid” or “plasmid vector” are used in reference to extra-chromosomal nucleic acid molecules capable of replication in a cell and to which an insert sequence can be operatively linked so as to bring about replication of the insert sequence. Vectors are used to transport DNA sequences into a cell, and some vectors may have properties tailored to produce protein expression in a cell, while others may not. A vector may include expression signals such as a promoter and/or a terminator, a selectable marker such as a gene conferring resistance to an antibiotic, and one or more restriction sites into which insert sequences can be cloned. Vectors can have other unique features (such as the size of DNA insert they can accommodate). A plasmid or plasmid vector is an autonomously replicating, extrachromosomal, circular DNA molecule (usually double-stranded) found mostly in bacterial and protozoan cells. Plasmids are distinct from the bacterial genome, although they can be incorporated into a genome, and are often used as vectors in recombinant DNA technology.

The term “expression vector” as used herein refers to a recombinant DNA molecule containing a desired coding sequence and appropriate nucleic acid sequences necessary for expression of the operably linked coding sequence (e.g., an insert sequence that codes for a product) in a particular host cell.

The term “epitope tag” or simply “tag” is meant to include, but not be limited to a GST (glutathione-S-transferase) tag, an HA (haemagglutinin) tag, a Myc tag, a FLAG tag, and a 6×His tag. The preceding listing of such epitope tag polypeptides is meant to be illustrative and not limiting, and there is a large and ever-increasing selection of such epitope polypeptides that are substitutable for substitution with those specifically described herein. One skilled in the art is capable of making desired substitutions without undue experimentation.

By “fusion” or “hybrid” protein, DNA molecule, or gene is meant a chimera of at least two covalently bonded polypeptides or DNA molecules.

As used herein, the term “origin of replication,” “origin,” or “ori” refers to a DNA sequence conferring functional replication capabilities in a host cell—it is a particular DNA sequence at which DNA replication is initiated and proceeds bidirectionally or unidirectionally. Examples include, but are not limited to, the SV40 replication origin, which is sufficient to promote DNA replication in animal cells and in vitro. An origin of replication may be a “high copy number” or “low copy number” origin of replication, and may exhibit a narrow or broad host range. There also exist significant differences between eukaryotic and prokaryotic origins of replication.

By “restriction endonuclease” and “restriction enzyme” is meant enzymes (e.g., bacterial enzymes), each of which cut double-stranded DNA at or near a specific nucleotide sequence (a cognate restriction site). Examples include, but are not limited to, KpnI, EcoRV, SfiI, XhoI, SalI, and NotI.

By “restriction” or “digestion” is meant cleavage of DNA by a restriction enzyme at its cognate restriction site.

By “restriction site” is meant a particular DNA sequence recognized by its cognate restriction endonuclease.

As used herein, the term “purified” or “to purify” refers to the removal of contaminants from a sample. For example, but without limitation, a 6×His affinity tag (comprising six consecutive histidine residues) binds with remarkable selectivity and affinity to certain matrices (e.g., a nickel-nitriloacetic acid (Ni-NTA) metal-affinity chromatography matrix). Thus, a protein engineered to bear a 6×His tag may be purified and isolated rapidly and efficiently.

As used herein, the terms “sequencing” or “DNA sequence analysis” refers to the process of determining the linear order of nucleotides bases in a nucleic acid sequence (e.g., insert sequence) or clone. These units are the C, T, A, and G bases. Generally, to sequence a section of DNA, the DNA sequence of a short flanking region, i.e., a primer binding site, must be known beforehand. One method for sequencing is called dideoxy sequencing (or Sanger sequencing). One example for performing dideoxy sequencing uses the following reagents: 1) the DNA that will be used as a template (e.g., insert sequence); 2) a primer that corresponds to a known sequence that flanks the unknown sequence; 3) DNA nucleotides, to synthesize and elongate a new DNA strand; 4) dideoxynucleotides that mimic the G, A, T, and C building blocks to incorporate into DNA, but that prevent chain elongation, thus acting as termination bases for a DNA polymerase (the four different dideoxynucleotides also may be labeled with different fluorescent dyes for automated DNA sequence analysis); and 5) a nucleic acid polymerizing agent (e.g., DNA polymerase or Taq polymerase, both of which are enzymes that catalyze synthesis of a DNA strand from another DNA template strand). When these reagents are mixed, the primer aligns with and binds the template at the primer binding site. The polymerizing agent then initiates DNA elongation by adding the nucleotide building blocks to the 3′ end of the primer. Randomly, a dideoxynucleotide will integrate into a growing chain. When this happens, chain elongation stops and, if the dideoxynucleotide is fluorescently labeled, the label will be also be attached to the newly generated DNA strand. Multiple strands are generated from each template, each strand terminating at a different base of the template. Thus, a population is produced with strands of different sizes and different fluorescent labels, depending on the terminal dideoxynucleotide incorporated as the final base. This entire mix may, for example, be loaded onto a DNA sequencing instrument that separates DNA strands based on size and simultaneously uses a laser to detect the fluorescent label on each strand, beginning with the shortest. The sequence of the fluorescent labels, read from the shortest fragment to the longest, corresponds to the sequence of the template. The reading may be done automatically, and the sequence may be captured and analyzed using appropriate software. The term “shotgun cloning” refers to the multi-step process of randomly fragmenting target DNA into smaller pieces and cloning them en masse into plasmid vectors.

As used herein, the terms “to clone,” “cloned,” or “cloning” when used in reference to an insert sequence and vector, mean ligation of the insert sequence into a vector capable of replicating in a host cell. The terms “to clone,” “cloned,” or “cloning” when used in reference to an insert sequence, a vector, and a host cell, refer generally to making copies of a given insert sequence. In this regard, to clone a piece of DNA (e.g., insert sequence), one would insert it into a vector (e.g., ligate it into a plasmid, creating a vector-insert construct) which may then be put into a host (usually a bacterium) so that the plasmid and insert replicate with the host. An individual bacterium is grown until visible as a single colony on nutrient media. The colony is picked and grown in liquid culture, and the plasmid containing the “cloned” DNA (the sequences inserted into the vector) is re-isolated from the bacteria, at which point there may be many millions of copies of the vector-insert construct. The term “clone” can also refer either to a bacterium carrying a cloned DNA, or to the cloned DNA itself.

The term “electrophoresis” refers to the use of electrical fields to separate charged biomolecules such as DNA, RNA, and proteins. DNA and RNA carry a net negative charge because of the numerous phosphate groups in their structure. Proteins carry a charge that changes with pH, but becomes negative in the presence of certain chemical detergents. In the process of “gel electrophoresis,” biomolecules are put into wells of a solid matrix typically made of an inert porous substance such as agarose. When this gel is placed into a bath and an electrical charge applied across the gel, the biomolecules migrate and separate according to size, in proportion to the amount of charge they carry. The biomolecules can be stained for viewing (e.g., with ethidium bromide or with Coomassie dye) and isolated and purified from the gels for further analysis. Electrophoresis can be used to isolate pure biomolecules from a mixture, or to analyze biomolecules (such as for DNA sequencing).

As used herein, the terms “PCR” and “amplifying” refer to the polymerase chain reaction method of enzymatically “amplifying” or copying a region of DNA. This exponential amplification procedure is based on repeated cycles of denaturation, oligonucleotide primer annealing, and primer extension by a DNA polymerizing agent such as a thermostable DNA polymerase (e.g., the Taq or Tfl DNA polymerase enzymes isolated from Thermus aquaticus or Thermus flavus, respectively.

As used herein, the term “RT-PCR” refers to reverse transcription polymerase chain reaction, which is used to amplify a particular segment of RNA. Using RT-PCR techniques, the RNA segment is first reverse transcribed into its DNA complement, and then the DNA complement is amplified using PCR.

As used herein, the term “oligonucleotide,” refers to a short length of single-stranded polynucleotide chain. Oligonucleotides are typically less than 100 residues long (e.g., between 15 and 50), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes. Oligonucleotides may be useful as PCR primers.

As used herein, the term “primer” refers to an oligonucleotide, whether occurring naturally as in a purified restriction digest or produced synthetically, which is capable of acting as a point of initiation of nucleic acid synthesis when placed under conditions in which synthesis of a primer extension product which is complementary to a nucleic acid strand is induced, (i.e., in the presence of nucleotides and an inducing agent such as DNA polymerase and at a suitable temperature and pH. The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

As used herein, the terms “PCR product,” “PCR fragment,” and “amplification product” refer to the resultant mixture of compounds after two or more cycles of the PCR steps of denaturation, annealing, and extension are complete. These terms encompass the case where there has been amplification of one or more segments of one or more target sequences.

The following examples are offered by way of illustration and not by way of limitation.

Based on available scientific knowledge for in vitro and in vivo biological activities of PSAP, Saposin C or active peptides derived from PSAP, the availability of recombinant human (“rh”) Saposin C and rh-PSAP would allow them to be used in the following disciplines: a) pain control and management; b) nerve regeneration following nerve-damage; c) myelin degenerative diseases; d) diabetic neuropathy, e) research in cell biology in general or, specifically, in vitro and in vivo cancer research; and f) reduce the loss of muscle mass following injury to the muscle's nerve supply.

Despite an increasing interest in PSAP and Saposin C in basic science research and its potential therapeutic applications in clinical medicine, there are only three reports of rh-Saposin C or rh-PSAP proteins: 1) rh-Saposin C expressed by infecting insect cells (Spodoptera Frugiperda Sf21) with a baculovirus harbouring human Saposin C cDNA (see, e.g., Gopalakrishnan M M, Grosch H W, Locatelli-Hoops S, Werth N, Smolenová E, Nettersheim M, Sandhoff K, Hasilik A. Purified recombinant human prosaposin forms oligomers that bind procathepsin D and affect its autoactivation. Biochem J. 383(Pt. 3), 507-515, 2004; PubMed ID: 15255780. Schultz-Heienbrok R, Remmel N, Klingenstein R, Rossocha M, Sandhoff K, Saenger W, and Maier T. Crystallization and preliminary characterization of three different crystal forms of human saposin C heterologously expressed in Pichia pastoris, Acta Crystallograph Sect F Struct Biol Cryst Commun. 62, 117-120, 2006; PubMed ID: 16511279.); 2) GST-tagged rh-PSAP (81.88 kDa) expressed in wheat germ cell-free protein synthesis system (Abnova, GmbH, Heidelberg, Germany, Cat# H00005660-P01), but the production level with this method is very low for molecules less than 80 kDa (www.abnova.com); and 3) rh-PSAP expressed in E. coli, a prokaryotic system (United States Biological, Inc., Swampscott, Mass., Cat# P9052-90; www.usbio.net).

The following points demonstrate the significance of the availability of our novel constructs and stable transfectants for mass production of rh-PSAP and rh-Saposin C.

pSecTag2A vectors subcloned with rh-PSAP or rh-Saposin C allows a convenient method of overexpressing the two proteins.

Human Saposin C is not (naturally an extracellular secreted molecule. By subcloning the full length human Saposin C DNA sequence into pSecTag2A vector, now it is possible to have extracellular secretion of the protein.

Moreover, expressing these proteins in a mammalian cell system (e.g., CHO-K1), as in the present invention, yields proteins that are appropriately glycosylated—unlike previously—isolated PSAP and Saposin C proteins expressed in non-mammalian cells (e.g., Sf9 insect cells or E. coli). The cells of nonhuman species do not glycosylate their proteins in the same way as human cells do. This is important because glycosylation profoundly affects biological activity, function, clearance from circulation, and (crucially) antigenicity.

Recombinant human Saposin C has never been produced for commercial purposes. The only available report of rh-Saposin C has the following characteristics: a) it is made only in an academic institution abroad (University of Bonn, Germany) and only for research purposes; b) it is made in insect cells (low expression level, non-mammalian eukaryotic cells) infected with a baculovirus containing 6×His-tagged human Saposin C cDNA; and c) it is non-glycosylated, or its glycosylation is not analogous to that of native Saposin C This lack of posttranslational modification might potentially limit some of the resulting protein's biological activities.

The other two rh-PSAP proteins (Commercially available from Abnova, Heidelberg, DE and from United States Biological, Inc., Massachusetts) are expressed in non-eukaryotic cells and so do not bear native mammalian posttranslational modifications—such as glycosylation—that may be important for their biological activities in mammalian cells. Naturally-expressed PSAP and Saposin C isolated from mammalian cells are heavily glycosylated. Finally, according to the manufacturers' data sheet, the biological activities of these rh-PSAP proteins are unknown.

Both rh-PSAP and rh-Saposin C constructs described in this invention report are fully characterized and demonstrate biological activities in human cell lines. These constructs could be independently available and could be used for transfection into any type of benign or malignant human cells for further basic science research or therapeutic applications.

In addition to the pSeqtag2A-rh-PSAP and pSecTag2A-rh-Saposin C constructs described here, we have also established and characterized stable-transfectants of CHO-K1 cells with the following characteristics: a) CHO-K1 stable transfectants express rh-PSAP or rh-Saposin C; b) these cells are immortal and could be propagated in available culture medium (with minimal growth requirements) and conveniently in small-scale in a laboratory or large-scale (industrial); c) both rh-PSAP and rh-Saposin C are tagged with hexa-histidine (6×His) sequence.

The 6×His sequence provides a convenient method for high quality purification and can also be used to trace the recombinant proteins after their use in in vivo (e.g., after injection into laboratory animals and following their tissue distribution or localization by immunohistochemical staining with anti-histidine antibody) or in vitro studies (e.g., after addition to cell culture, its localization on the cell membrane or involvement with intracellular trafficking could be investigated by different methods such as immunofluoresence staining). Based on these characteristics, we have used and we herein present a methodology that provides a reliable and convenient method for purification of final proteins expressed by the cells.

The applications for the pSecTag2A-rh-PSAP and pSecTag2A-rh-Saposin C constructs and stable CHO-K1-transfectants stably expressing soluble rh-PSAP and rh-Saposin C are for basic science research and clinical therapy in the field of cancer, neuropathies (e.g., diabetic neuropathies), pain control and management, nerve regeneration (after damage to nervous system), diseases related to muscles (following hypoxia or injury to muscles or their nerve supply), myelin degenerative disorders (e.g., multiple sclerosis), neuroembryological diseases, neuronal differentiation, neurochemistry, and neurobiology.

Here, for the first time we report a continuous source, high-level, and stable expression of recombinant human Saposin C and PSAP with a C-terminal hexa-histidine (6×His) tag in Chinese hamster ovary (CHO-K1) cells that are recognized as the most popular mammalian host for commercial production of proteins. 6×His-tagged rh-Saposin C and 6×His-tagged rh-PSAP were purified from the culture medium of stably transfected CHO cells using an anti-His affinity gel. In addition, we demonstrate the biological activities of these proteins functional assays (cell proliferation and motility), using human prostate stromal cells and cancer cells.

The differences between rh-PSAP and rh-Saposin C described in the prior art is summarized in TABLE 1 below.

TABLE 1
Characteristics of rh-PSAP and rh-Saposin C from different systems

| Protein | | Expression | | M.W. |
|---|---|---|---|---|
| (reference) | 6 × His | System | Glycosylation | (kDa) |
| rh-PSAP | Yes | CHO-K1 | Yes; high level | 68-72 |
| (SEQ ID NO: 17) | | | | |
| rh-PSAP (1) | No | Baculovirus- | Yes; low level | 58 |
| | | infected Sf9 | | |
| rh-PSAP (2) | No | E. coli | No | 81.88 |
| rh-PSAP (3) | Yes | E. coli | No | Unknown |
| rh-Saposin C | Yes | CHO-K1 | Yes | 10 |
| (SEQ ID NO: 18) | | | | |
| rh-Saposin C (4) | Yes | E. coli | No | 8 |
| rh-Saposin C (5) | Yes | Baculovirus- | Yes; low level | 8 |
| | | infected Sf9 | | |

In TABLE 1, the rh-PSAP from reference (1) is described in Hiraiwa M, O'Brien J S, Kishimoto Y, Galdzicka M, Fluharty A L, Ginns E I, Martin B M. Isolation, characterization, and proteolysis of human prosaposin, the precursor of saposins (sphingolipid activator proteins). Arch Biochem Biophys. 304, 110-116, 1993; the rh-PSAP from reference (2) is commercially available from Abnova Co., Taiwan, Cat No. H00005660-P01, and which also bears a GST tag; and the rh-PSAP from reference (3) is commercially available from United States Biological, Inc., Swampscott, Mass., Cat# P9052-90; www.usbio.net.

In TABLE 1, the rh-Saposin C from reference (4) is described in Qi X, Leonova T, Grabowski G A. Functional human saposins expressed in Escherichia coli. Evidence for binding and activation properties of saposins C with acid beta-glucosidase. J Biol Chem. 269, 16746-16753, 1994; and the rh-Saposin C from reference (5) is described by Sandhoff K., Unpublished data, From the Institut für Organische Chemie und Biochemie, Universität Bonn, D-53121 Bonn, Germany.

### Example 1

### Cell Lines and Cell Culture

Human prostate stromal cells (Pr.St; Catalogue No. CO-2608) were purchased from BioWhittaker (Walkersville, Md.). The cells were maintained according to the manufacturer's instructions in a defined culture medium specific for each cell type, named “Pr.EGM” (Catalogue No. CC3166) and “SCGM” (Catalogue No. CC-3205) for prostate epithelial and stromal cell lines, respectively. A human prostate cancer cell line, PC-3 was purchased from the ATCC (Cat # CRL-1435) and cultured routinely in Dulbeccos's Modified Eagle's Medium (DMEM) supplemented with 10% heat-inactivated fetal bovine serum (HI-FBS). Chinese hamster ovary cell line (CHO-K1: ATCC, Manassas, Va., USA, Cat No. CCL-61) and 293T/17/17 human embryonic kidney cell lines (ATCC, Cat # CRL-11268) were purchased from ATCC. CHO-K1 cells were routinely cultured in Kaighn's modification of Ham's F12 medium (F12K) supplemented with 2 mM L-glutamine, 1.5 g/L sodium bicarbonate, and 10% FBS. The 293/T17 cell line was maintained in DMEM with 4 mM L-glutamine, 4.5 g/L glucose, 1.5 g/L sodium bicarbonate, and supplemented with 10% FBS.

Baculovirus expressed recombinant human Saposin C (Bac-rh-Saposin q and goat anti-human Saposin C antibody were provided as gifts from Professor K. Sandoff (Bonn, Germany), and were characterized by ELISA, immunoblot, and immunoprecipitation. Purified human milk-PSAP (PSAP purified from human milk) and mouse monoclonal anti-PSAP antibody were gifts from Professor Masao Hiraiwa (University of California, San Diego) and have been characterized previously for Western blotting and other techniques.

### Example 2

### Construction of rh-PSAP and rh-Saposin C Expression Vectors

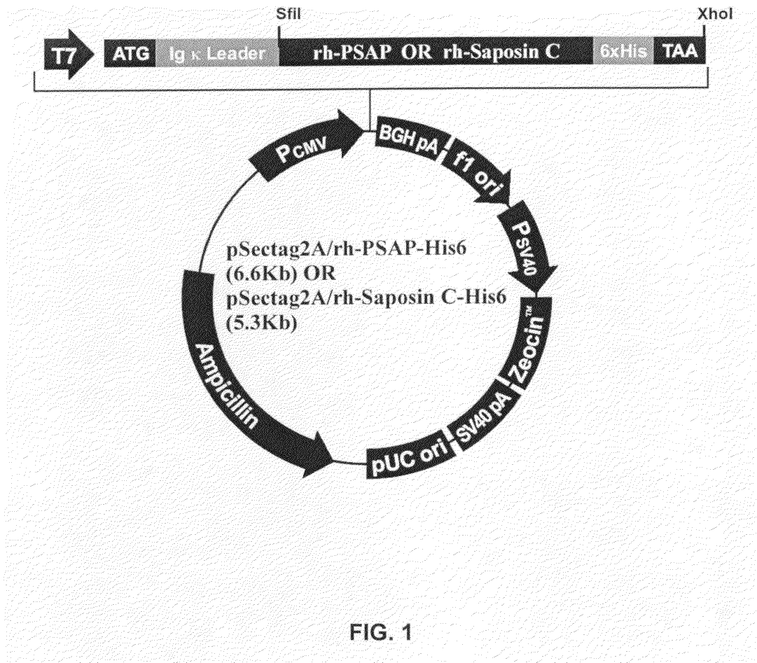

Prosaposin is the precursor protein of saposins A, B, C, D with 524 amino acids including a 16 amino acids signal peptide (SP) which leads the secretion of mature prosaposin into culture supernatant. However heterogeneous signal peptides, such as Ig kappa-chain signal peptide, have been shown more efficient for secreted expression of recombinant proteins in mammalian cells. See, e.g., Coloma M J, Hastings A, Wims L A, Morrison S L. Novel vectors for the expression of antibody molecules using variable regions generated by polymerase chain reaction. J Immunol Methods. 52, 89-104, 1992; PubMed ID: 1640112. We first tested if the secreted expression level of rh-PSAP in the pSecTag2A vector (Invitrogen, Carlsbad, Calif., USA, Cat No. V900-20) with the murine Ig kappa-chain signal peptide will be higher than in the pcDNA3.1 vector (Invitrogen, Carlsbad, Calif., USA, Cat No. V790-20) having the prosaposin native signal peptide. In addition, we tested whether adding prosaposin native signal peptide after the Ig kappa signal peptide in pSecTag2A vector would increase the secretion efficiency of recombinant prosaposin. As shown in FIG. 1, proteins expressed from pSecTag2 vectors are fused at the N-terminus to the murine Ig kappa-chain leader sequence for protein secretion and at the C-terminus to a peptide containing six tandem histidine residues for detection and purification. The Zeocin™ resistance gene of the pSecTag2 vectors (FIG. 1) permits selection in both E. coli and mammalian cells in the presence of the antibiotic Zeocin™ (a formulation of phleomycin D1, a copper-chelated glycopeptide antibiotic produced by Streptomyces CL 990, available from InvivoGen, San Diego, Calif.). The pSecTag2 vector exists as versions A, B, and C, to facilitate correct in-frame fusion with the Ig kappa-chain leader sequence.

To construct rh-PSAP and rh-Saposin C expression vectors, total RNA was extracted from normal human prostate epithelial (Pr.EP) cells (Biowhittaker, Walkersville, Md., USA) with RNeasy Mini Kit (Qiagen, Maryland, USA, Cat No. 74014). The full-length PSAP cDNA was synthesized by RT-PCR with Affinity Script Multi Temperature cDNA Synthesis Kit (Stratagene, TX, USA, Cat No. 200436). Specific primers were designed to introduce a hexa-histidine (6×His) tag to the c-terminus of PSAP or Saposin C to facilitate recombinant protein purification, which are listed in TABLES 2 and 3, below. All primers were synthesized by Integrated DNA Technology (IDT, Inc: Coralville, Iowa).

TABLE 2
Primers for rh-PSAP and rh-Saposin C expression vectors

| | | | |
|---|---|---|---|
| Vector | Name | Length | Primer Sequence |

| | | | |
|---|---|---|---|
| pcDNA3.1 | PSAP-1F | 32 | 5′-CGGGGTACCACCATGTACGCCCT |
| (rh-PSAP-His6) | Forward | | CTTCCTCCT-3′ |
| | | | (SEQ ID NO: 10) |
| | PSAP-1B | 63 | 5′-TATGATATCCTAATGGTGATGGTG |
| | Backward | | ATGGTGGTTCCACACATGGCGTTTG |
| | (6 × His tagged) | | CAATGCTCGACAGC-3′ |
| | | | (SEQ ID NO: 11) |
| pSecTag2A | PSAP-2F | 36 | 5′-AAAGCGGCCCAGCCGGCCGGCCC |
| (rh-PSAP-His6 | Forward | | GGTCCTTGGACTG-3′ |
| or | | | (SEQ ID NO: 12) |
| rh-PSAP-SP-His6) | PSAP-SP-2F | 37 | 5′-AAAGCGGCCCAGCCGGCCATGTA |
| | Forward | | CGCCCTCTTCCTCC-3′ |
| | | | (SEQ ID NO: 13) |
| | PSAP-2B | 50 | 5′-CCGCTCGAGCTAGTGATGGTGATGG |
| | Backward | | TGATGGTTCCACACATGGCGTTTGC-3′ |
| | | | (SEQ ID NO: 14) |
| pSecTag2A | Saposin C-F | 39 | 5′-AAAGCGGCCCAGCCGGCCTCTGAT |
| (rh-Saposin C-His6) | Forward | | GTTTACTGTGAGGTG-3′ |
| | | | (SEQ ID NO: 15) |
| | Saposin C-B | 49 | 5′-CCGCTCGAGCTAGTGATGGTGATG |
| | Backward | | GTGATGCGTGCCAGAGCAGAGGTGC-3′ |
| | | | (SEQ ID NO: 16) |

To construct pcDNA3.1-rh-PSAP-His6 vector (1-524aa, with native signal peptide), the gene for rh-PSAP was amplified by PCR using the above cDNA template and sub-cloned into KpnI and EcoRV sites of the pcDNA3.1 vector. To construct pSecTag2A-rh-PSAP-His6 (17-524aa, without native signal peptide), pSecTag2A/rh-PSAP-SP-His6 (1-524aa, with native signal peptide (SP)), or pSecTag2A-rh-Saposin C-His6 (311-391aa, without native signal peptide) vectors (SEQ ID NO:8 & SEQ ID NO:9, respectively), the gene for rh-PSAP or rh-Saposin C was amplified by PCR using the above cDNA template (from the pcDNA3.1-rh-PSAP-His6 vector) with Phusion High-Fidelity DNA Polymerase (New England Biolabs, Beverly, Mass., USA, Cat No. F530S) and specific primers with sequences compatible for digestion with restriction enzymes (SfiI+XhoI) and subcloning into pSecTag2A vector (TABLES 2 & 3).

TABLE 3
Summary of rh-PSAP and rh-Saposin C expression vectors

| | | | | PSAP | | | |
|---|---|---|---|---|---|---|---|
| | PSAP | | | native | Ig-kappa | | |
| Vector | Gene | Forward | Backward | signal | Signal | 6xHis | |
| Name | (aa) | Primer | Primer | peptide | peptide | Tag | Cloning Sites |
| pcDNA3.1- | 1-524aa | PSAP-1F | PSAP-1B | Yes | No | Yes | KpnI + EcoRV |
| rh-PSAP- | | | | | | | |
| His6 | | | | | | | |
| pSecTag2A- | 17-524aa | PSAP-2F | PSAP-2B | No | Yes | Yes | SfiI + XboI |
| rh-PSAP- | | | | | | | |
| His6 | | | | | | | |
| pSecTag2A- | 1-524aa | PSAP- | PSAP-2B | Yes | Yes | Yes | SfiI + XboI |
| rh-PSAP- | | SP-2F | | | | | |
| SP-His6 | | | | | | | |
| pSecTag2A- | 311-391aa | SAPC-F | SAPC-B | No | Yes | Yes | SfiI + XboI |
| rh-Saposin | | | | | | | |
| C-His6 | | | | | | | |

The PCR products were gel-purified by DNA Gel Extraction Kit (Qiagen, Maryland, USA, Cat No. 28704) and digested with SfiI (New England Biolabs, MA, USA, Cat No. R0123S) and XhoI (New England Biolabs, Beverly, Mass., USA, Cat No. R0146S), yielding rh-PSAP insert and rh-Saposin C insert. The inserts were ligated by T4 DNA ligase (Invitrogen, Carlsbad, Calif., USA, Cat No. 15224) into pSecTag2A vector which was digested with the same enzymes, and transformed into One Shot E. coli Top10 Competent cells (Invitrogen, Carlsbad, Calif., USA, Cat No. C7510-03). To construct vector, all plasmids were purified by Qiaprep Spin Miniprep Kit (Qiagen, Maryland, USA, Cat No. 27106). The above three vectors harboring rh-PSAP (SEQ ID NO:6) and rh-Saposin C (SEQ ID NO:7) cDNA were subjected to DNA sequencing in both directions (5′ to 3′ and 3′ to 5′) and proved to be 100% accurate and equal to native PSAP and Saposin C sequence (ACGT Inc, Wheeling, Ill., USA).

### Example 3

### Transient Transfection and Expression of rh-PSAP in 293T/17 Cells

To select the best vector for rh-PSAP expression, we first expressed rh-PSAP in Human Embryonic Kidney (HEK) 293T/17 cells by transient transfection. 293T/17 cell line has been engineered to constitutively express the temperature sensitive gene for simian virus 40 (SV40) T-antigen, allowing for episomal replication of the plasmids which contain the SV40 origin of replication sequence and highly transient expression of recombinant proteins.

293T/17 cell line (ATCC, Manassas, Va., USA, Cat No. CRL-11268) was cultured in Dulbecco's Modified Eagle Medium (DMEM, Gibco, Grand Island, N.Y., USA, Cat No. 11595) supplemented with 10% Heat Inactivated Fetal Bovine Serum (HI-FBS, Gibco, Auckland, NZ, USA, Cat No. 10082) and 1% penicillin/streptomycin (P/S) antibiotics (Sigma, St. Louis, Mo., USA, Cat No. A5955) in a humidified 5% CO2 incubator at 37° C. 5×10⁵ cells were seeded in 60 mm dishes and cultured overnight to 40%-60% confluency. 2 μg DNA was mixed with 10 μl Lipofectin reagent (Invitrogen, Carlsbad, Calif., USA, Cat No. 18292-037) in Opti-MEM Reduced Serum Medium (“Opti-MEM,” Gibco, Auckland, NZ, USA, Cat No. 31985) for 16 hour transfection. The cells were replaced with 2 ml fresh Opti-MEM reduced serum medium and cultured for additional 48 hours.

The culture supernatants (2.5 ml) were centrifuged at 1500 rpm for 5 minutes to remove cellular debris and concentrated 10-fold to final 250 μl with 2 ml Vivaspin Centrifuge Tube with 10 kDa cut-off membrane (Vivascience, Stonehouse, UK, Cat No. VS0602). Total protein concentration in supernatant was measured by the BCA Protein Assay Kit (Pierce, Rockford, Ill., USA, Cat No. 23225). A total of 10 μg protein supernatant for each transfection were separated in 4-20% Novex Tris-Glycine Polyacrylamide Gel (Invitrogen, Carlsbad, Calif., USA, Cat No. EC6028BOX), and then electrotransferred to Hybond PVDF membrane (Amersham, Little Chalfont, UK, Cat No. RPN303F). The membranes were blocked with 5% BSA (Santa Cruz, Calif., USA, Cat No. SC-2323) in TBST buffer (20 mM Tris HCl, 150 mM NaCl, 0.1% Tween 20) for 1 hour at room temperature. The membranes were first incubated with mouse anti-PSAP antibody as the primary antibody (1:500 dilution in 0.2% BSA in TBST) and then incubated with goat anti-mouse IgG-horseradish peroxidase-conjugated secondary antibody (1:2000 dilution in 0.2% BSA in TBST, Santa Cruz, Calif., USA, Cat No. SC-2005) each for 1 hour at room temperature. The blots were then detected by ECL Plus reagents (Amsersham, Little Chalfont, UK, Cat No. RPN2109).

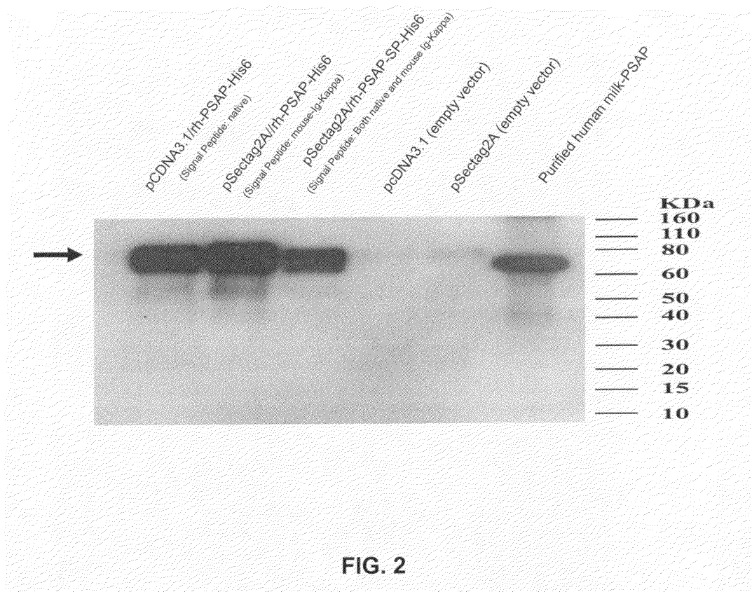

As shown in FIG. 2, 293T/17 cells transiently transfected with the pSecTag2A-rh-PSAP-His6 vector (with murine Ig-kappa signal peptide, FIG. 2 lane 2) demonstrated a higher level of PSAP protein expression (secretion) in culture supernatant than 293T/17 cells transiently transfected with the pcDNA3.1-rh-PSAP-His6 (with native signal peptide, FIG. 2, lane 1) or pSecTag-2A-rh-PSAP-SP-His6 vector (with both native signal peptide and murine Ig-kappa signal peptide, FIG. 2, lane 3). This data suggested that the murine Ig-kappa signal peptide sequence is more efficient in leading the secretion of recombinant PSAP than its native signal peptide or a combination of both signal peptides. As a control, 293T/17 cells transfected with empty vectors (pcDNA3.1 or pSecTag2A, FIG. 2, lanes 4 and 5, respectively) secreted very low levels of endogenous human prosaposin. However, transient transfection of 293T/17 cells with pSecTag/2A-rh-Saposin C-His6 tag did not reveal a detectable rh-Saposin C expression in the culture supernatant (data not shown).

### Example 4

### CHO-K1 Transfection and Selection of Stable Cell Lines

Although the 293T/17 cell line proved a useful model cell line for rh-PSAP expression with either pcDNA3.1 or pSecTag2A vectors harboring PSAP-cDNA, this cell line was only good and feasible for transient expression of small quantities of recombinant proteins. In addition, using transient transfection in this cell line did not lead to a detectable rh-Saposin C protein in concentrated culture supernatant. In order to bypass these technical limitations and to achieve a reliable method for consistent production of large quantities of pure rh-PSAP and rh-Saposin C proteins for biological and potential clinical therapeutic usage, we chose the CHO-K1 cell line (see, e.g., American Type Culture Collection No. CCL-61). This cell line is one of the most popular mammalian hosts for stable expression of recombinant proteins especially for clinical and therapeutic applications. See, e.g., Andersen D C, Krummen L. Recombinant protein expression for therapeutic applications. Curr Opin Biotechnol. 13, 117-123, 2002; PubMed ID: 11950561. Warnock J N, Al-Rubeai M. Bioreactor systems for the production of biopharmaceuticals from animal cells. Biotechnol Appl Biochem. 45(Pt 1), 1-12, 2006; PubMed ID: 16764553. So far there is no report about the expression of rh-PSAP and rh-Saposin C in CHO-K1 cell line. We used the vector pSecTag2A/rh-PSAP-His6 for the transfection of CHO-K1 cells and selection of stable cell lines.

For transfection, 3×10⁵ cells were seeded in 60 mm dishes and cultured overnight to 20-30% confluency. 4 μg DNA of pSecTag2A/rh-PSAP-His6 or pSecTag2A/rh-Saposin C-His6 vector was mixed with 20 μl Lipofectin reagent in Opti-MEM reduced serum medium for 16 hours transfection. The cells were changed to Ham's F-12K medium (Mediatech, through Fisher Scientific) supplemented with 10% FBS and 1% Penicillin/Streptomycin, and cultured for an additional 48 hours to reach near confluency.

The cells in 60 mm dishes were collected by 0.25% Trypsin-EDTA (Gibco, Grand Island, N.Y., USA, Cat No. 25200). 20% of the cells were diluted to ten 100 mm dishes in F-12K selective medium supplemented with 10% HI-FBS, 1% Penicillin/Streptomycin and 500 μg/ml Zeocin (Invitrogen, Carlsbad, Calif., USA, Cat No. 45-0430). At the fourth day, the cells were changed to the same fresh selective medium. At the seventh day, a single colony was isolated with cloning cylinders (Sigma, St. Louis, Mo., USA, Cat No. C3983) and expanded in 24-well plates and then in T25 flasks in the same selective medium. It took about another 7-10 days for the cells in T25 flasks to reach greater than 90% confluency. The cells were then collected and split into one T75 flask (to make cell stock) and another T25 flask (to measure the relative productivity rh-PSAP and rh-Saposin C by Western blotting). For cell stock, after the cells in T75 flask were 90% confluency, the cells were collected by trypsin, re-suspended in F-12K medium supplemented with 20% HI-FBS, 1% Penicillin/Streptomycin and 10% DMSO (Sigma, St. Louis, Mo., USA. Cat No. D2650) in several tubes, and then stored in liquid nitrogen.

After the cells in the new T25 flask reached 90% confluency, they were washed twice with phosphate-buffered saline (PBS) and replaced with 2 ml Opti-MEM reduced serum medium supplemented with 1% penicillin/streptomycin. After culture for 24 hours, the supernatants were collected and 10 μg supernatant protein for each clone was subjected to SDS-PAGE and Western blotting as in Example 3 and FIG. 2. Goat anti-Saposin C polyclonal antibody (1:100 dilution in 0.2% BSA in TBST) was used as primary antibody. This antibody can detect both human Saposin C and PSAP. Donkey anti-goat IgG-horseradish peroxidase-conjugated antibody (1:2000 dilution in 0.2% BSA in TBST, Santa Cruz, Calif., USA, Cat No. SC-2020) was used as secondary antibody.

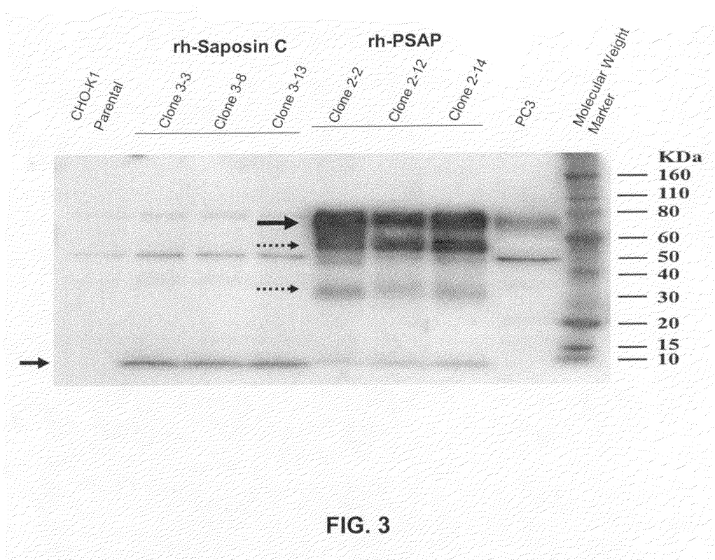

The expression results are shown in FIG. 3. We obtained three rh-PSAP highly expressive stable CHO-K1 clones, which are named as Clone 2-2, Clone 2-12, and Clone 2-14 from a total of fifteen Zeocin-resistant pSecTag2A/rh-PSAP-His6 clones. We also obtained three rh-Saposin C highly expressive stable CHO-K1 clones, which are named as Clone 3-2, Clone 3-8, and Clone 3-13 from a total of fourteen Zeocin-resistant pSecTag2A/rh-Saposin C-His 6 clones. Among them, Clone 2-2 and Clone 3-13 were finally used for large-scale expression and purification of rh-PSAP (SEQ ID NO:17) and rh-Saposin C (SEQ ID NO:18), respectively.

### Example 5

### Large-Scale Expression and Purification of rh-PSAP and rh-Saposin C

For a typical large-scale expression and purification, two tubes of Clone 3-13 or Clone 2-2 cell stock were recovered from liquid nitrogen and amplified in T150 flasks in 30 ml of F-12K medium supplemented with 10% FBS, 1% Penicillin/Streptomycin and 250 μg/ml Zeocin to near confluency (about three days). The cells were then split into eight T150 flasks and cultured to near confluency (about three days). The cells were then split into seventeen T500 Triple Flasks (Nunc, Roskilde, Denmark, Cat No. 132920) containing 60 ml of F-12K medium supplemented with 10% FBS, 1% Penicillin/Streptomycin and 250 μg/ml Zeocin. After reaching 90% confluency, the cells were washed twice with PBS and replaced with 60 ml Opti-MEM reduced serum medium supplemented with 1% Penicillin/Streptomycin. After 48 hours, the culture supernatant (about 1 liter) was collected in two 500 ml conical centrifuge bottles (Corning, N.Y., USA, Cat No. 431123) by centrifugation at 1500 rpm for 5 minutes at 4° C. to remove cellular debris. The cleared supernatant was stored at −80° C. for further purification.

Recombinant human PSAP and Saposin C were purified by a batch-absorption method using Ni-NTA Superflow Resins (Qiagen, Maryland, USA, Cat No. 1018611). One liter supernatant was mixed with 100 ml neutralization buffer (0.5 M NaPi, pH 8.0, 1.5 M NaCl, 2 mM imidazole), centrifuged at 15,000×g for 10 minutes at 4° C., filtered through 0.22 μm membrane (Corning, N.Y., USA, Cat No. 430758), and pooled in two 500 ml conical centrifuge bottles. The pooled filtered supernatant was incubated with 5 ml Ni-NTA resin overnight at 4° C. or for 4 hours at room temperature by slow gyroscopic spin at 100 rpm. The resin was subsequently collected and loaded onto an empty 5 ml polypropylene purification column (Qiagen, Maryland, USA, Cat No. 34964). To remove non-specifically bound proteins, the resin was first washed four times with 5 ml of binding buffer (50 mM Na₂HPO₄, pH 8.0, 300 mM NaCl) containing 10 mM imidazole (Sigma, St. Louis, Mo., USA, Cat No. 11102-1000) and then washed four times with 5 ml of binding buffer containing 20 mM imidazole. The Ni-NTA resin-bound rh-PSAP or rh-Saposin C were eluted four times with 2.5 ml of binding buffer containing 50 mM imidazole and then four times with 2.5 ml of binding buffer containing 100 mM imidazole. For each washing and elution, the 2.5 ml fraction was collected into one tube.

The appropriate fractions (about 10-15 ml) containing the purified recombinant protein were pooled, and buffer-exchanged twice to PBS via ultrafiltration with Vivaspin centrifugal concentrators (Sartorius Stedim Biotech S.A., France). For rh-PSAP, 6 ml Vivaspin concentrator with 10 kDa cut-off membrane (Vivascience, Stonehouse, UK, Cat No. VS0602) was used for buffer exchange and condensation. For rh-Saposin C with an approximate molecular weight of 10 kDa a 6 ml centrifuge tube with 50 kDa cut-off membrane (Vivascience, Stonehouse, UK, Cat No. VS0631) was first used to remove non-specific proteins of high molecular weight by collecting the flow-through, and then 15 ml centrifuge tube with 2 kDa cut-off membrane (Vivascience, Stonehouse, UK, Cat No. VS15RH91) was used for buffer exchange and condensation. After two rounds of buffer exchange, the final imidazole concentration was less than 3 mM. The purified proteins were quantified by measuring the absorbance at OD 280 nm using serial dilutions of BSA as standards. The purified rh-PSAP and rh-Saposin C were filtered through 0.22 μm filters and stored at −80° C. for future use without detectable loss of biological activities. Several methods were used to characterize the purified rh-PSAP and rh-Saposin C, including SDS-PAGE with Coomassie Blue R-250 Staining (Bio-Rad, Hercules, Calif., Cat No. 161-0436), Silver Staining Plus (Bio-Rad, Hercules, Calif., Cat No. 161-0449), and Western blotting with mouse anti-PSAP antibody or goat anti-Saposin C polyclonal antibody.

For Coomassie Blue R-250 staining, culture supernatants and imidazole eluted fractions for rh-PSAP and rh-Saposin C were separated in a 4-20% gel. After SDS-PAGE, the gel was incubated with 50 ml Coomassie Blue R-250 Staining Solution (Bio-Rad, Hercules, Calif., Cat No. 161-0436) for 1 hour with gentle shaking at room temperature. Then the gel was incubated with 50 ml de-staining buffer (45% methanol and 10% acetic acid in double-distilled (“dd”) H₂O). The de-staining buffer was changed several times, until the gel showed clear protein bands and background staining was satisfactorily eliminated.

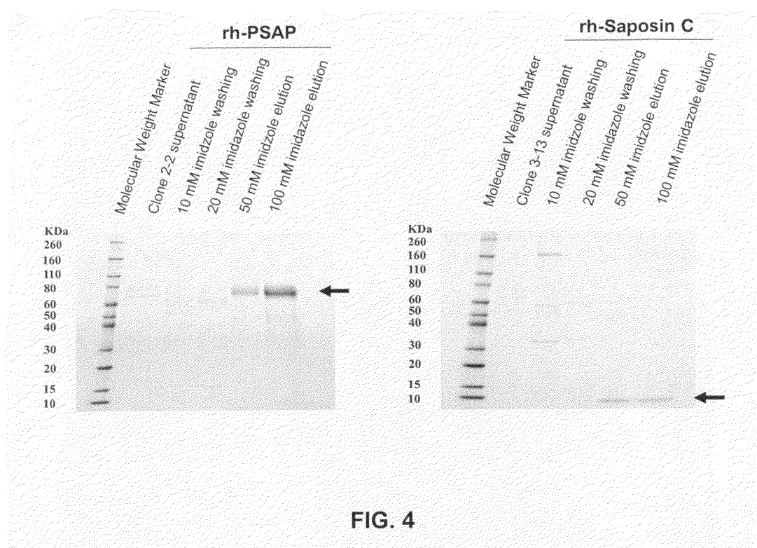

The Coomassie blue staining results for rh-PSAP and rh-Saposin C purification are shown in FIG. 4. Most non-specific bound proteins were washed away by 10 mM and 20 mM imidazole washing. Purified rh-PSAP and rh-Saposin C were completely eluted by 50 mM and 100 mM imidazole washing steps. The purified rh-PSAP showed a molecular weight of approximately 72 kDa, which is the same size as native human prosaposin secreted by PC3 prostate cancer cells. The purified rh-Saposin C showed a single band of approximately 10 kDa. For a typical purification from 1 L supernatant, the yield of rh-PSAP was 1.5 to 3 mg/L, and the yield of rh-Saposin C was 0.25 to 0.5 mg/L.

For silver staining, we used the Silver Staining Plus Kit (Bio-Rad, Hercules, Calif., Cat. No. 161-0449). After SDS-PAGE, the gel was fixed with 200 ml fixative enhancer solution (50% methanol, 10% acetic acid, 10% fixative enhancer concentrate, 30% ddH₂O) for 30 minutes, washed twice with 200 ml ddH₂O, and then stained with 100 ml staining and developing solution (5 ml silver complex solution, 5 ml reduction moderator solution, 5 ml image development reagent, 50 ml development accelerator solution, 35 ml ddH₂O) for 10-15 minutes until the gel showed clear protein bands. The reaction was stopped with 100 ml of 5% acetic acid for 10 minutes.

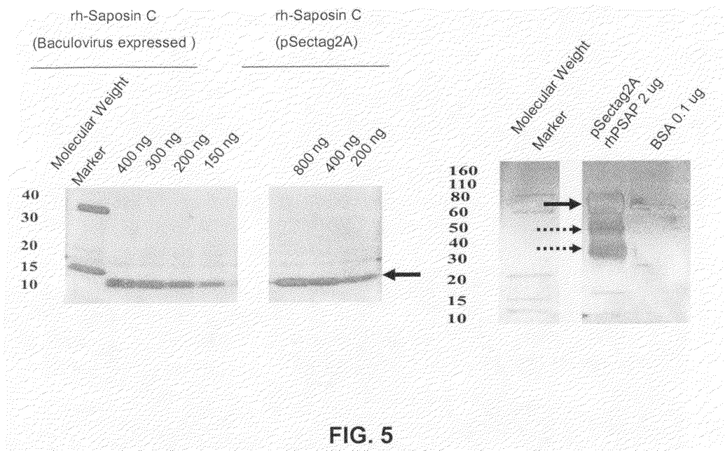

The silver staining results for rh-PSAP and rh-Saposin C are shown in FIG. 5. The purity of rh-PSAP and rh-Saposin C was estimated to be above 95% according to the silver staining results. The purified rh-PSAP showed two additional bands at approximately 35 and 55 kDa. These bands represent proteolytically-cleaved products of extracellular PSAP in the form of various tri-saposins or di-saposins. This type of intracellular or extracellular fragmentation of PSAP is a common observation and has been reported in native protein isolated from human seminal plasma and breast milk or rh-PSAP expressed in baculovirus-infected insect cells and their culture medium, as well as in incubations of purified PSAP with cathepsin D enzyme. See, e.g., Hiraiwa M, O'Brien J S, Kishimoto Y, Galdzicka M, Fluharty A L, Ginns E I, Martin B M. Isolation, characterization, and proteolysis of human prosaposin, the precursor of saposins (sphingolipid activator proteins). Arch Biochem Biophys. 304, 110-116, 1993; PubMed ID: 8323276. Kondoh K, Hineno T, Sano A, Kakimoto Y. Isolation and characterization of prosaposin from human milk Biochem Biophys Res Commun. 181, 286-292, 1991; PubMed ID: 1958198. Henseler M, Klein A, Glombitza G J, Suzuki K, Sandhoff K. Expression of the three alternative forms of the sphingolipid activator protein precursor in baby hamster kidney cells and functional assays in a cell culture system. J Biol. Chem. 271, 8416-8423, 1996; PubMed ID: 8626540.

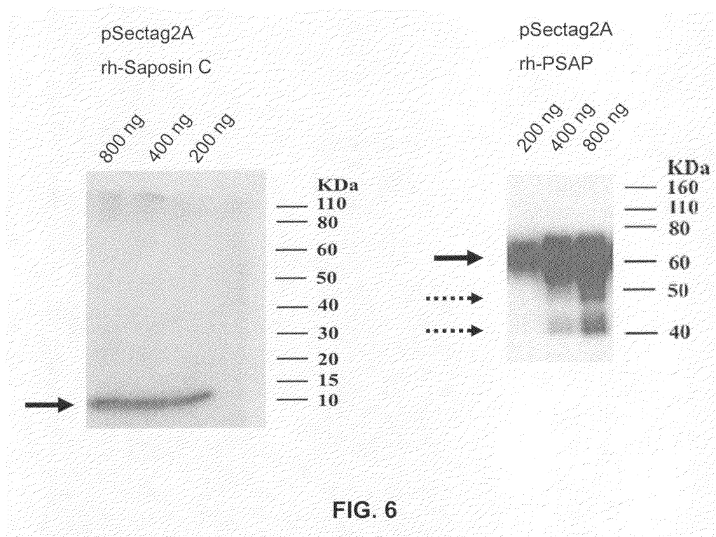

Western blotting was done as in EXAMPLE 3 (“Transient transfection and expression of rh-PSAP in 293T/17 cells”). Briefly, purified rh-PSAP and rh-Saposin C were subjected to SDS-PAGE and then transferred to PVDF membrane. After BSA blocking, mouse anti-PSAP and goat anti-Saposin C were used to blot rh-PSAP and rh-Saposin C, respectively. The Western blotting results for rh-PSAP and rh-Saposin C are shown in FIG. 6.

### Example 6

### Biological Activity of Purified rhPSAP and rhSAPC CHO-K1 Purified Recombinant Human-Saposin C and PSAP Stimulate Prostate Stromal Cell Proliferation

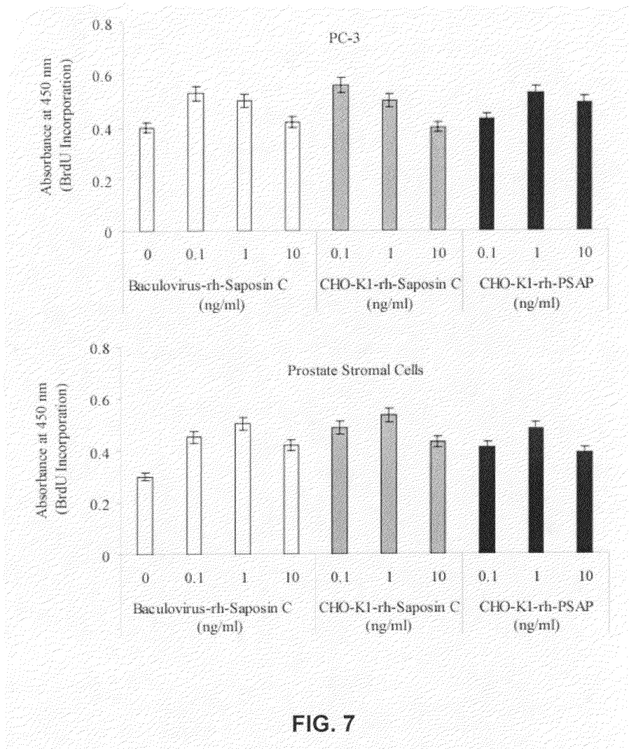

To examine the biological activities of the recombinant proteins expressed and purified here (rh-Saposin C and rh-PSAP), we tested their effect on bromodeoxyuridine (BrdU incorporation into newly synthesized DNA. BrdU can be incorporated into the newly-synthesized DNA of replicating cells, substituting for thymidine during DNA replication. Detection of BrdU in these cells thus indicates that the cells were actively replicating their DNA, and so were actively proliferating. We used a BrdU Cell Proliferation Assay Kit, following the manufacturer's instructions (The Exalpha Biologicals, Inc, Maynard, Mass., Cat# X1327K2). As a positive (active) control, we used rh-Saposin C from the baculovirus expression system in insect cells. PC3 and prostate stromal cells were seeded in 96-well plate at 1000 cells/well in their growth medium. After 3 days, they were incubated either in serum-free basal medium or medium supplemented with either rh-Saposin, or rh-PSAP at concentrations of 0.1, 1.0, 10, or 100 ng/ml. After incubation for 4 days and refreshing the recombinant protein after the first 2 days, BrdU incorporation was determined according to the kit manufacturer's instructions. The results are shown in FIG. 7, in which each point represents the mean±SEM of 8 replicates from a representative experiment of three independently-repeated experiments.

As we expected, baculovirus expressed rh-Saposin C increased BrdU incorporation into newly synthesized DNA up to 32% in PC3 cells and up to 66% in PrSt cells (FIG. 7). In a dose dependent manner, rh-Saposin C stimulated BrdU incorporation into newly synthesized DNA by 19-33% in PC3 cells and by 43-61% in PrSt cells. Finally, rhPSAP increased BrdU incorporation by 7-32% in PC3 cells and by 30-60% in PrSt cells.

### Example 7

### Biological Activity of Purified rhPSAP and rhSAPC CHO-K1 Purified rh-Saposin C and rh-PSAP Stimulate Prostate Stromal and Cancer Cell Motility

To test the effect of the pSecTag2A-expressed recombinant proteins rh-Saposin C and rh-PSAP on PC3 and PrSt cell motility, a cell migration assay was performed using 24-well transwell units with 8-μm polycarbonate filters (Costar, Cat # 3422, Becton Dickinson, Bedford, Mass.). For the experiment, the lower compartment of each transwell unit contained 400 μl of basal media supplemented with 2% FBS and 0.1% BSA. Baculovirus-expressed rh-Saposin C (as a positive control), rh-Saposin C, or rh-PSAP were added to the lower compartment at the following concentrations: 0 (control), 0.1 ng/ml, 1 ng/ml, 10 ng/ml, or 100 ng/ml.

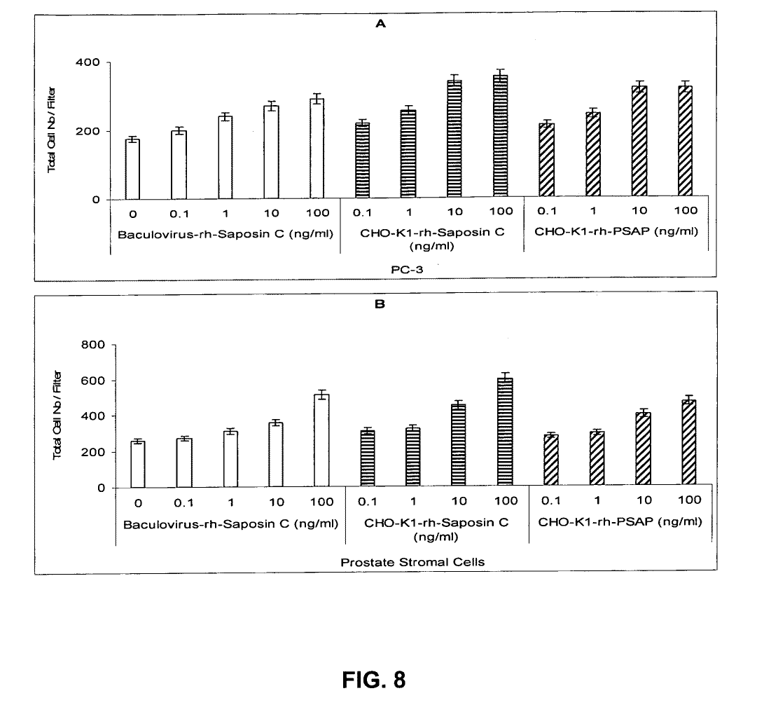

Cultured cells were freshly harvested by trypsinization and counted. Approximately 5×10⁴ cells for PC3 cell line and 1.5×10⁴ cells for PrSt cells were resuspended in 200 μl of serum-free medium with 0.1% BSA, and were placed in the upper compartment of the transwell unit. After 24 hours of incubation at 37° C., cells in both the upper and lower compartments were fixed in methanol and stained with Diff-Quick (Dade, Aguada, Puerto Rico). Non-migratory cells in the upper surface of the filter were removed by wiping with a cotton swab. Cells under the filter were counted. The results are shown in FIG. 8. Each bar represents the mean±SEM of 8 replicates from a representative experiment of three independently repeated experiments.

PrSt cell migratory response to recombinant proteins was more pronounced than the response seen in PC-3 cells. As expected, baculovirus-expressed rh-Saposin C in a dose-dependent manner increased cell migration in PC-3 cells by 14-64% and in PrSt cells by 3-96%. As seen in FIG. 8, rh-Saposin C and rh-PSAP increased PrSt cells migration by 19-130% and 8-80%, respectively. In PC-3 cells, rh-Saposin C and rh-PSAP increased cell migration by 25-100% and 20-81%, respectively. These data indicate that both pSecTag2A-expressed/purified rh-Saposin C and rh-PSAP from CHO-K1 clones are capable of eliciting a biological response of cell motility in mammalian cells and are thus properly considered biologically active molecules.

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only, the scope of the present invention is to be limited only by the following claims.

The invention claimed is:

1. A cell transfected with a mammalian expression vector, wherein said vector provides for expression of rh-PSAP, and wherein said rh-PSAP is extracellularly secreted.

2. The cell of claim 1, wherein said cell is a CHO-K1 cell.

3. The cell of claim 2, wherein said transfection is stable transfection.

4. The cell of claim 3, wherein said vector bears, in 5′ to 3′ direction, an Ig kappa-chain V-J2-C signal peptide sequence, SEQ ID NO:6, and a hexa-histidine epitope.

5. The cell of claim 3, wherein said vector is a pSecTag2 vector into which SEQ ID NO:6 has been cloned.

6. The cell of claim 3, wherein said vector is SEQ ID NO:8.

7. A cell transfected with a mammalian expression vector, wherein said vector provides for expression of rh-Saposin C, and wherein said rh-Saposin C is extracellularly secreted.

8. The cell of claim 7, wherein said cell is a CHO-K1 cell.

9. The cell of claim 8, wherein said transfection is stable transfection.

10. The cell of claim 9, wherein said vector bears, in 5′ to 3′ direction, an Ig kappa-chain V-J2-C signal peptide sequence, SEQ ID NO:7, and a hexa-histidine epitope.

11. The cell of claim 9, wherein said vector is a pSecTag2 vector into which SEQ ID NO:7 has been cloned.

12. The cell of claim 9, wherein said vector is SEQ ID NO:9.

13. An isolated polypeptide comprising the amino acid sequence of SEQ ID NO:17.

14. The polypeptide of claim 13, wherein said polypeptide is an N-linked glycoprotein.

15. The polypeptide of claim 14, wherein said polypeptide is a sialylated N-linked glycoprotein.

16. An isolated polypeptide comprising the amino acid sequence of SEQ ID NO:18.

17. The polypeptide of claim 16, wherein said polypeptide is an N-linked glycoprotein.

18. The polypeptide of claim 17, wherein said polypeptide is a sialylated N-linked glycoprotein.
